(19) United States
(12) Patent Application Publication
(10) Pub. No.: US 2023/0255860 A1
(43) Pub. Date: Aug. 17, 2023

(54) PAINT MATERIAL COMPOSED OF COSMETIC, DILUENT FOR PAINT MATERIAL, PAINT, AND PAINT SET

(71) Applicant: MANGATA Co., Ltd., Tokyo (JP)

(72) Inventors: Hisanori TANAKA, Tokyo (JP); Mayuri TANAKA, Tokyo (JP)

(73) Assignee: MANGATA Co., Ltd., Tokyo (JP)

(21) Appl. No.: 17/998,387

(22) PCT Filed: Apr. 30, 2021
(86) PCT No.: PCT/JP2021/017230

§ 371 Date: Nov. 10, 2022

(30) Foreign Application Priority Data
May 29, 2020 (JP) 2020-094103

(51) Int. Cl. A61K 8/19; A61K 8/02; A61Q 1/10; A61Q 1/12; A61Q 1/08

(52) U.S. Cl. CPC A61K 8/19; A61K 8/022; A61Q 1/10; A61Q 1/12; A61Q 1/08

(57) ABSTRACT

The paint material composed of the cosmetic according to the present invention has the minimum characteristics as the paint by being used in combination with the diluent for the paint material, and can be equivalently used as the typical paint, and an effective use of the cosmetic to be discarded can be achieved. In particular, by using such diluent in combination, it is possible to expect favorable characteristics peculiar to the paint material based on the cosmetic in terms of a transparency, a quick-drying, a smell, and the like of a film when stretched and spread on a drawing paper as compared with the typical paint.

### TECHNICAL FIELD

The present invention relates to a paint material composed of a cosmetic, a diluent for the paint material, a paint, and a paint set. More specifically, the present invention relates to the paint material composed of a cosmetic that can use a cosmetic and the like to be discarded, the diluent for the paint material, the paint, and the paint set.

### BACKGROUND ART

The cosmetic including powdery cosmetic is distributed, and is mainly used by females. Typical powdery cosmetic includes a base makeup such as a foundation and a face powder, and a point makeup such as an eye shadow and a blusher, and these are all used for purposes of smearing on human skin or the like. In addition, most of powders such as extender powders constituting the powdery cosmetic are derived from minerals (see, for example, Patent Literature 1), but some minerals are faced with a problem of depletion.

On the other hand, individual consumers often discard the powdery cosmetic without using it up, and companies that manufacture and sell cosmetic also discard a large amount of bulk (contents) of the powdery cosmetic that have not cleared a certain quality in a year. Even in view of a recent situation in which a sustainable society should be aimed at, an effective use of the powdery cosmetic to be discarded should be considered, and an establishment of a technology corresponding thereto is desired.

### CITATION LIST

### Patent Literature

Patent Literature 1: JP 2003-012450 A

### SUMMARY OF INVENTION

### Technical Problem

Here, in many cases, a pigment or a bright powder is blended in the cosmetic such as the powdery cosmetic, and in this case, there is a characteristic that a wide variety of colors can be visually enjoyed. If the powdery cosmetic to be discarded can be converted into, for example, a paint while having related characteristics, a new use method of the powdery cosmetic to be discarded can be provided.

Generally, an oily component (an oil solution) such as a silicone oil or a hydrocarbon oil, extender powders such as a mica, a talc, a silica, a barium sulfate, or a synthetic phlogopite, a coloring material such as a titanium dioxide, and a surfactant or the like as necessary are blended in the powdery cosmetic, and these are often packed and pressed in a container such as a gold dish.

For this reason, there is a case where the powder in the powdery cosmetic is partially covered with the oily component of a certain amount, and even when the powdery cosmetic is pulverized and kneaded with a commercially available aqueous liquid, a wetting does not proceed and the powdery cosmetic is separated, so that it is difficult to form the powdery cosmetic into a slurry like a paint.

The present invention has been made in view of the above, and it is an object of the present invention to provide the paint material composed of a cosmetic that can be used in the same manner as a commercially available typical watercolor paint, the diluent for the paint material, the paint, and the paint set.

### Solution to Problem

In order to solve the above problems, the paint material composed of the cosmetic according to the present invention includes:

the extender powders and the coloring material, and

when contained components are arranged in a descending order of a content, the cosmetic having three or less oily components in the top five places is included.

In the present invention described above, when the contained components are arranged in the descending order of the content, the paint material composed of the cosmetic according to the present invention includes one or more of the extender powders in the top three places.

In regard to the paint material composed of the cosmetic according to the present invention, in the present invention described above, the extender powders included in the top three places are at least one selected from the group consisting of the mica, the talc, the synthetic phlogopite, the silica, and the barium sulfate.

In regard to the paint material composed of the cosmetic according to the present invention, in the present invention described above, a pearl powder is not included in the top three places when the contained components are arranged in the descending order of the content.

In regard to the paint material composed of the cosmetic according to the present invention, in the present invention described above, the cosmetic is at least one selected from the group consisting of the eye shadow, the blusher, a body powder, the face powder, a bleaching powder, a skincare powder, a loose powder, a presto powder, and the foundation.

The diluent for the paint material according to the present invention is the diluent for diluting the paint material composed of the cosmetic and includes components below:

a water of 49.5 to 89.0 mass % with respect to the entire diluent;

a lower alcohol of 10.0 to 35.0 mass % with respect to the entire diluent; and

a polyvinyl alcohol of 0.5 to 15.0 mass % with respect to the entire diluent.

In regard to the diluent for the paint material according to the present invention, in the present invention described above, the lower alcohol is an ethanol.

The paint according to the present invention includes

a mixture obtained by mixing:

the paint material composed of the cosmetic of the present invention described above; and

the diluent for the paint material according to the present invention described above.

The paint set according to the present invention includes:

the paint material composed of the cosmetic of the present invention described above; and

the diluent for the paint material according to the present invention described above.

### Advantageous Effects of Invention

When used in combination with the diluent for the paint material, the paint material composed of the cosmetic according to the present invention has the minimum properties as the paint and can be used in the same manner as a typical paint, which can achieve the effective use of the cosmetic to be discarded. In particular, by using such diluent in combination, it is possible to expect good characteristics peculiar to the paint material based on the cosmetic in terms of a transparency, a quick-drying property, a smell, and the like of a film when the paint material is stretched and spread on a piece of drawing paper as compared with the typical paint.

When the diluent for the paint material according to the present invention is used in combination with the paint material composed of the cosmetic, the diluent for the paint material can play a role of compensating for the component insufficient as the paint material with respect to the cosmetic for the paint material.

The paint and the paint set according to the present invention include the paint material composed of the cosmetic according to the present invention and the diluent for the paint material according to the present invention, and can be provided as the paint that can be used in the same manner as the typical paint and as the paint set in which the paint material and a dedicated diluent are included.

### BRIEF DESCRIPTION OF DRAWINGS

FIG. 1 is a diagram showing evaluation criteria used in Test Example 1 and Test Example 2.

### DESCRIPTION OF EMBODIMENTS

Hereinafter, one aspect of the present invention will be described.

### (I) Paint Material Composed of Cosmetic

Although the present invention is the paint material composed of the cosmetic, examples of the cosmetic that is considered to be applicable to the present invention among the commercially available cosmetics include, the cosmetic having the powdery cosmetic such as various eye shadows (eye colors) that include a powder eye shadow (an eye color), various blushers (cheek colors) that include a powder blusher, the body powder, the face powder, the bleaching powder, the skincare powder, the loose powder, the presto powder, and makeup cosmetics (makeup) that include the foundation; and a cosmetic having the powdery cosmetic such as base cosmetics serving as a base of the makeup cosmetic that include a cosmetic base.

These cosmetics may be used alone, or may be used in combination of two or more thereof. In addition, there is no problem even if these cosmetics to be discarded are used, and by using the cosmetic originally scheduled to be discarded, the effective use of the cosmetic to be discarded can be achieved.

Among these, the eye shadow (the eye color) is, for example, the cosmetic that is applied to form a shadow by attaching colors of different tones to eye holes on eyelids side and a periphery of eyes such as lower eyelids and outer corners of the eyes.

The blusher (the cheek color) is a so-called cheek rouge (a blush) and is a cosmetic used to make one's complexion look bright by applying to a cheek or to create a three-dimensional effect by shading the cheek. A dosage form may be a solid form, a powder form, a cream form, a liquid form, a stick form, and the like, and the powder form is suitable for the present invention.

The body powder is a cosmetic used for suppressing a glare of oil and giving a natural finish with a mat.

The face powder is a cosmetic used for finishing makeup and for suppressing a so-called gleaming (shininess) and stickiness of sebum and the foundation. The bleaching powder having a whitening effect, the skincare powder having a skincare effect, and the loose powder are also considered to be one kind of the face powder and the body powder.

A breast powder is a kind of the face powder described above, and is basically used after using the foundation described later. It is a cosmetic for conditioning and coating the foundation to make one's skin look more beautiful.

The foundation is a cosmetic used to condition the color of the skin or to cover freckles, stains, and the like of the skin to protect the skin from ultraviolet rays. It is the makeup cosmetic to be applied after applying the cosmetic base described later on the skin.

The cosmetic base is used as a base before applying the makeup cosmetic such as the foundation, and is a cosmetic as a base material to be used for the purpose of bettering a color development property, an adhesion force (an adhesion), and a spreadability (a covering) of the makeup cosmetic, preventing a beauty of a makeup finish and a makeup deterioration, and maintaining a makeup effect.

Additionally, the powdery cosmetic suitable for the paint material according to the present invention generally refers to the cosmetic that has the powder (the powder component) as a main component (it is considered that the content is mostly 60 mass % or more of the entire cosmetic, and is preferably 60 to 95 mass %, but is not limited thereto) and is appropriately mixed with an aqueous component, the oily component, or the like as necessary, and such powdery cosmetic is understood to refer to the cosmetic having powder particles with a particle diameter of approximately 1.0 mm or less and allowing each particle to freely flow at a normal temperature and a normal pressure, but is not particularly limited thereto and can widely use the cosmetic generally called the powdery cosmetic in the cosmetic industry.

Examples of the component contained in the cosmetic to be an object represented by the above include the extender powders (for example, an inorganic powder, an organic powder, a surfactant metal salt powder (metallic soap), and the like), the coloring material (for example, an inorganic pigment, an organic pigment, a natural pigment, the pearl powder, and the like), the oily component (for example, natural vegetable oils and animal oils, the hydrocarbon oil, an ester oil (including a glyceride oil), a higher fatty acid, a higher alcohol, the silicone oil, a wax, an oil thickener, an organic solvent, and the like), and other components (for example, an aqueous preservative, a powder preservative, a preservative, a surfactant, a surface treatment agent, an antioxidant, a cosmetic agent, an ultraviolet absorber, a film-forming agent, an emollient component, an aqueous component, and the like). These components may be used alone, or may be used in combination of two or more thereof. Further, similarly for each of the components listed below, they may be used alone, or may be used in combination of two or more thereof. Among them, the extender powders and the coloring material are essential components for applying the cosmetic as the paint material, but the oily component and the other components are not particularly limited as described above. Further, as for the oily component, as will be described later, when the contained components in the cosmetic are arranged in the descending order of the content, the number of types of the oily component is set to three or less among the top five places.

Generally, examples of the powder contained in the powdery cosmetic include the extender powders such as the inorganic powder, the organic powder, and the surfactant metal salt powder (the metallic soap), and the coloring material such as the inorganic pigment (including a white pigment), the organic pigment, the natural pigment, and the pearl powder. Here, the powders listed below may or may not be subjected to various surface treatments.

It is understood that the extender powder (also called as the extender pigment) is used as an extender for maintaining the dosage form of the cosmetic, and is also used as an adjuster for accommodating an extensibility, an adhesiveness, a gloss, and the like or adjusting a color tone of the cosmetic.

The component used as the extender powders is the inorganic powder or a pulverized product of a naturally derived clay mineral. In addition, for example, a synthetic inorganic powder such as a silicon dioxide (the silica), the organic powder such as an organic polymer powder which is not the inorganic pigment, the surfactant metal salt powder (the metallic soap), and the like may also be individually classified.

In cosmetic, the extender powder is also used for the purpose of preventing the makeup deterioration due to an absorption of sweat and sebum, improving the feel of use by adjusting a powder shape, the particle size, a hardness, and a surface state, adjusting the extensibility or the adhesiveness of the cosmetic, adjusting a whiteness and the color tone, and adjusting the gloss of the cosmetic by controlling the transparency, and for example, the extender powder is the essential component as a base component for the foundation or the like.

Examples of the inorganic powder as the extender powders include a zirconium oxide, a cerium oxide, a magnesium oxide, a barium sulfate (a sulfate Ba), a calcium sulfate, a magnesium sulfate, a calcium carbonate, a magnesium carbonate, the talc, the mica, a kaolin, a sericite, a muscovite, a synthetic mica, the synthetic phlogopite (a synthetic fluorofurogopite), a phlogopite, a red mica, a biotite, a lepidolite, a silicic acid, an anhydrous silicic acid, an aluminum silicate, a magnesium silicate, an aluminum magnesium silicate, a calcium silicate, a barium silicate, a strontium silicate, the silicic acid (Na/K/Al), a tungstic acid metal salt, a hydroxyapatite, a vermiculite, a hydrargilite, a bentonite, a montmorillonite, a hectorite, a disteardimonium hectorite, a zeolite, a ceramic powder, a dibasic calcium phosphate, an alumina, an aluminium hydroxide, a boron nitride, a BN, the silica, a fine particle cerium oxide, a dimethylsilylated silica, an aluminum corn starch octenyl succinate (an aluminum corn starch octenylsuccinate), an aluminum starch octenyl succinate (an aluminum starch octenylsuccinate), and the like.

Examples of the organic powder include a polyamide powder, a polyester powder, a polyethylene powder (which also becomes a wax), a polypropylene powder, a polystyrene powder, a polyurethane powder, a benzoguanamine powder, a polymethylbenzoguanamine powder, a polytetrafluoroethylene powder, a polymethylmethacrylate powder, a cellulose powder, a silk powder, a nylon powder (a nylon 12, a nylon 6, and the like), and the like. Note that these descriptions of the “powder” are for convenience, and also include those not described as “powder” (for example, “polyethylene,” “polypropylene,” and the like).

Examples of other organic powders include a styrene-acrylic acid copolymer, a divinylbenzene-styrene copolymer, a vinyl resin, a urea resin, a phenol resin, a fluororesin, a silicone resin, an acrylic resin, a melamine resin, an epoxy resin, a polycarbonate resin, a microfine crystalline fiber powder, a starch powder, a lauroyl lysine (which also serves as the surface treatment agent), a polymethylsilsesquioxane, a (vinyl dimethicone/methicone silsesquioxane) crosspolymer, a (dimethicone/vinyl dimethicone) crosspolymer, a (diphenyl dimethicone/vinyl diphenyl dimethicone/silsesquioxane) crosspolymer, a polymethyl methacrylate, a methyl methacrylate crosspolymer, a corn starch, a hydrogenated starch hydrolysate, and the like.

Examples of the surfactant metal salt powder (the metallic soap) include a zinc stearate, an aluminum stearate, an aluminum distearate (an Al distearate), a calcium stearate, a magnesium stearate (an Mg stearate), a zinc myristate, a magnesium myristate (an Mg myristate), a zinc cetyl phosphate, a calcium cetyl phosphate, a sodium zinc cetyl phosphate, and the like.

The coloring material is a powder or the like for coloring the cosmetic into a desired color and developing the color, and examples of the inorganic pigment serving as the pigment as the coloring material include inorganic white pigments such as fine particles and a pigment grade (a titanium dioxide (a rutile), the titanium dioxide (an anatase), and the like) titanium oxide (which is the white pigment and also becomes the pearl powder), fine particles and a pigment grade zinc oxide (which is the white pigment and also serves as the powdery preservative), inorganic red pigments such as an iron oxide, an iron hydroxide and an iron titanate, inorganic brown pigments such as γ-iron oxide, inorganic yellow pigments such as a yellow iron oxide and yellow earth, inorganic black pigments such as a black iron oxide and a carbon black, inorganic purple pigments such as a manganese violet and a cobalt violet, inorganic green pigments such as a chromium hydroxide, a chromium oxide, a cobalt oxide and a cobalt titanate, inorganic blue pigments such as a ferric ferrocyanide (a Prussian blue) and an ultramarine (an ultramarine blue), and the like.

As the organic pigment which is a pigment, for example, one obtained by laking a tar-based pigment, one obtained by laking the natural pigment, a synthetic resin powder obtained by combining these powders, and the like can be used, and examples thereof include a red 2, a red 3, a red 102, a red 104, a red 105, a red 106, a yellow 4, a yellow 5, a green 3, a blue 1, a blue 2, a red 201, a red 202, a red 203, a red 204, a red 205, a red 206, a red 207, a red 208, a red 213, a red 214, a red 215, a red 218, a red 219, a red 220, a red 221, a red 223, a red 225, a red 226, a red 227, a red 228, a red 230(1), a red 230(2), a red 231, a red 232, an orange 201, an orange 203, an orange 204, an orange 205, an orange 206, an orange 207, a yellow 201, a yellow 202(1), a yellow 202(2), a yellow 203, a yellow 204, a yellow 205, a green 201, a green 202, a green 204, a green 205, a blue 201, a blue 202, a blue 203, a blue 204, a blue 205, a brown 201, a purple 201, a red 401, a red 404, a red 405, a red 501, a red 502, a red 504, a red 505, a red 506, an orange 401, an orange 402, an orange 403, a yellow 401, a yellow 402, a yellow 403, a yellow 404, a yellow 405, a yellow 406, a yellow 407, a green 401, a green 402, a blue 403, a blue 404, a purple 401, a black 401, and the like.

Examples of the natural pigment as the coloring material include a carmine, a carminic acid, a laccaic acid, a carthamin, a brazilin, a crocin, a salol yellow, a β-carotene, a hibiscus pigment, a capsainthin, a laccai acid, a gurcumin, a riboflavin, a shikonin, and the like.

The pearl powder being the coloring material may be a powder having a pearl gloss which is generally widely used, and in addition to usual mica titaniums with the pearl gloss, examples thereof include an iron oxide coated mica titanium, an iron oxide coated mica, a black iron oxide coated mica titanium, an iron oxide and black iron oxide coated mica titanium, a carmine coated mica titanium, a carmine and ferric ferrocyanide coated mica titanium, an iron oxide and carmine treated mica titanium, a ferric ferrocyanide treated mica titanium, an iron oxide and ferric ferrocyanide treated mica titanium, a chromium oxide treated mica titanium, a black titanium dioxide treated mica titanium, the synthetic phlogopite, a titanium dioxide coated synthetic phlogopite, a glass powder, a titanium dioxide coated glass powder, a polyethylene terephthalate polyester film laminated powder, mica flakes, an acrylic resin coated aluminum powder (which may contain the coloring material), a titanium dioxide coated mica, a titanium dioxide coated bismuth oxychloride, a titanium dioxide coated talc, a colored titanium dioxide coated mica, a bismuth oxychloride, a fish scale foil, aluminum flakes, stainless steel flakes, a titanium dioxide coated plate-shaped alumina, a tin oxide (tin), pearl shells, a gold foil, a gold vapor deposited resin film, a metal vapor deposited resin film, and the like.

Further, examples of the pearl powder other than this include the titanium dioxide (which also becomes the white pigment), a borosilicate (Ca/Al), a borosilicate (Ca/Na), (PET/polymethyl methacrylate) laminate, (PET/Al) laminate, (PET/Al/epoxy resin) laminate, and the like.

The oily component (also called the oil solution) contains an oil content and is useful for making the dosage form smooth, and examples thereof include the natural vegetable oils and animal oils, or the like, the hydrocarbon oil, the ester oil (including the glyceride oil), the higher fatty acid, the higher alcohol, the silicone oil, the wax, the oil thickener, the organic solvent, and the like.

Examples of the natural vegetable oils and animal oils or the like as the oily component include an avocado oil, a linseed oil, an almond oil, an ibota wax, an eno oil, an olive oil, a cacao butter, a kapok wax, a kaya oil, a carnauba wax, a cod-liver oil, a candelilla wax, an apricot kernel oil, a hydrogenated oil, a wheat germ oil, a sesame oil, a rice germ oil, a rice bran oil, a sugarcane oil, a sasanqua oil, a safflower oil, a shea butter, an evening primrose oil, a cinnamon oil, a jojoba wax, a shellac wax, a turtle oil, a soybean oil, a tea seed oil, a camellia oil, an Oenothera biennis oil, a corn oil, a rapeseed oil, a Japanese tung oil, a rice bran wax, a germ oil, a persic oil, a palm oil, a palm kernel oil, a castor oil, a hydrogenated castor oil, a castor oil fatty acid methyl ester, a sunflower oil, a grape oil, a bayberry wax, a jojoba oil, a macadamia nut oil, a mink oil, a cottonseed oil, a cotton wax, a Japan wax, a Japan mokurou kernel oil, a montan wax, a coconut oil, a hydrogenated coconut oil, a tri-coconut oil fatty acid glyceride, a peanut oil, a lanolin, a liquid lanolin, a reduced lanolin, a lanolin alcohol, a hard lanolin, a lanolin acetate, a lanolin fatty acid isopropyl, a POE lanolin alcohol ether, a POE lanolin alcohol acetate, a lanolin fatty acid polyethylene glycol, a POE hydrogenated lanolin alcohol ether, a hydrogenated vegetable oil, an egg yolk oil, and the like.

Examples of the hydrocarbon oil include a squalane, a squalene, a paraffin, a paraffin wax, a liquid paraffin (a mineral oil), a pristane, a polyisobutylene, a petrolatum, a polyisobutene, a polybutene, and the like.

Examples of the ester oil (including the glyceride oil) include a diisobutyl adipate, a 2-hexyldecyl adipate, a di-2 heptylundecyl adipate, a N-alkyl glycol monoisostearate, an isocetyl isostearate, a trimethylolpropane triisostearate, an ethylene glycol di-2 ethylhexanoate, a hexadecyl 2 ethylhexanoate, a glyceryl tri(2-ethylhexanoate) (a triethylhexanoin), a trimethylolpropane tri-2 ethylhexanoate, a pentaerythritol tetra-2 ethylhexanoate, a cetyl octanoate, an octyldodecylgum ester, an oleyl oleate, an octyldodecyl oleate, a decyl oleate, a neopentyl glycol dicaprate, a triethyl citrate, a 2-ethylhexyl succinate, an amyl acetate, an ethyl acetate, a butyl acetate, an isocetyl stearate, a butyl stearate, a diisopropyl sebacate, a di-2-ethylhexyl sebacate, a cetyl lactate, a myristyl lactate, an isopropyl palmitate, a 2-ethylhexyl palmitate (an ethylhexyl palmitate), a 2-hexyldecyl palmitate, a 2-heptylundecyl palmitate, a cholesteryl 12-hydroxystearate (a cholesteryl hydroxystearate), a dipentaerythritol fatty acid ester, an isopropyl myristate, an octyldodecyl myristate, a 2-hexyldecyl myristate, a myristyl myristate, a hexyldecyl dimethyloctanate, an ethyl laurate, a hexyl laurate, an N-lauroyl-L-glutamic acid-2-octyldodecyl ester, a diisostearyl malate, an acetoglyceryl, a glyceryl triisooctanoate, a polyglyceryl-2 triisostearate (a glyceryl triisostearate) (which is also used as the surfactant), a glyceryl triisopalmitate, a glyceryl monostearate, a glyceryl di-2 heptylundecanoate, a glyceryl trimyristate, a diglyceryl isostearate myristate, a tritridecyl trimellitate, a hexa(hydroxystearic acid/stearic acid/rosin acid) dipentaerythrityl, a propylene glycol dicaprate (a PG dicaprate), an isotridecyl isonanoate, a glyceryl tri-(caprylic acid/capric acid), an isononyl isononanoate, a bisdiglyceryl polyacyl adipate-2, a neopentyl glycol diethylhexanoate, an octyldodecyl stearoyloxystearate, an isostearyl neopentanoate, a hydrogenated castor oil isostearate, a hydrogenated tri-fatty acid (C12-18) glyceryl, and the like.

Examples of the higher fatty acid include a lauric acid, a myristic acid, a palmitic acid, a stearic acid, a behenic acid, an undecylenic acid, an oleic acid, a linoleic acid, a linolenic acid, an arachidonic acid, an eicosapentaenoic acid (EPA), a docosahexaenoic acid (DHA), an isostearic acid, and a 12-hydroxystearic acid (hydroxystearic acid).

Examples of the higher alcohol include a lauryl alcohol, a myristyl alcohol, a palmityl alcohol, a stearyl alcohol, a behenyl alcohol, a hexadecyl alcohol, an oleyl alcohol, an isostearyl alcohol, a hexyldodecanol, an octyldodecanol, a cetostearyl alcohol, a 2-decyltetradecinol, a cholesterol, a phytosterol, a POE cholesterol ether, a monostearyl glycerin ether (a butyl alcohol), a monooleyl glyceryl ether (a selachyl alcohol), and the like.

Examples of the silicone oil include a dimethyl silicone oil, a cyclic dimethyl silicone oil, a methyl hydrogen silicone oil, a polyether-modified silicone oil, a methyl phenyl silicone oil, a fluorine-modified silicone oil, a dimethicone, a hydrogen dimethicone, a cyclopentasiloxane, a methicone, a diphenylsiloxyphenyl trimethicone, a phenyl trimethicone, and the like.

Examples of the wax include the candelilla wax, a microcrystalline wax, a polyethylene (which also becomes the extender powders), a ceresin, an ozokerite, a beeswax, a synthetic wax, a hydrogenated palm oil fatty acid, a stearyl stearate, and the like.

Examples of the oil thickener include an (ethylene/propylene/styrene) copolymer, a (butylene/ethylene/styrene) copolymer, an (ethylene/propylene) copolymer, a dextrin palmitate, and the like.

Examples of the organic solvent include an isododecane and the like.

Examples of the aqueous preservative for other components include a phenoxyethanol, a caprylyl glycol, and the like. Examples of the powdery preservative include a zinc oxide (which also becomes the white pigment) and the like. Examples of the preservative include a butylene glycol (BG, which also becomes the aqueous component) methyl paraben, an ethyl paraben, a propyl paraben, a butyl paraben, a caprylyl glycol, an ethylhexylglycerin, a propanediol, a sodium dehydroacetate (an Na dehydroacetate), a silver, and the like.

Examples of the surfactant include a sorbitan sesquiisostearate, the polyglyceryl-2 triisostearate (which is also used as the oily component), a sorbitan stearate, a polyhydroxystearic acid, a polyglyceryl polyricinoleate-3 (which is also used as the oily component), and the like.

Examples of the surface treatment agent include a perfluorohexylethyltriethoxysilane, the isostearic acid, the stearic acid, the aluminium hydroxide (the Al hydroxide), a triethoxycaprylylsilane, the palmitic acid, a lauroyllysine (which also becomes the extender powder), and the like.

Examples of the antioxidant include a dibutylhydroxytoluene (BHT), a tocopherol, a tocopherol acetate, a diethylhexyl syringlidene malonate, and the like.

Examples of the cosmetic agent include a lecithin, a chamomile extract, a strawberry fruit extract, a lavender flower extract, a maggot root bark extract, a water-soluble collagen, a chitosan, a lactobacilli/hibiscus flower fermentation liquid, a rhodomyrtus tomentosa fruit extract, a camellia seed oil, a hydrolyzed conchiolin, a di(phytosteryl/octyldodecyl) lauroyl glutamate (a ceramide), and the like.

Examples of the ultraviolet absorber include an ethylhexyl methoxycinnamate, a t-butyl methoxydibenzoylmethane, the ethylhexyl methoxycinnamate, and the like.

Examples of the film-forming agent include a trimethylsiloxysilicic acid and the like.

Examples of the emollient component include a tri(caprylic acid/capric acid)glyceryl and the like.

Examples of the aqueous component include the butylene glycol (BG, which also becomes the preservative), water, and the like.

The above cosmetic applicable to the present invention preferably contains the coloring material, and when the contained components are arranged in the descending order of the content, the cosmetic preferably contains one or more of the extender powders in the top three places. Since the coloring material contained in the cosmetic often has a brightness and the extender powder also serves as a base material of the paint material and often assists the color development, the coloring material is suitable for use as the paint material and various colors can be visually enjoyed by using the extender powder as the essential component. In addition, the extender powder has a function of accommodating a hiding power, a coloring power, a covering power, a spreadability, the adhesiveness, and the like when the extender powder is spread on the drawing paper as the paint.

In addition, the coloring material is mostly contained in an amount of preferably 0.05 mass % or more and particularly preferably 2.0 to 30.0 mass % with respect to the entire cosmetic, but the coloring material is not particularly limited to this range as long as it has the color development suitable for the use as the paint material. Further, in a case where an insufficient color development or the like is felt when the cosmetic is applied as the paint material, the coloring material may be separately added to obtain the content described above.

While the extender powder contained in the cosmetic is suitable as the paint material as described above, in the case where one or more of the extender powders are contained in the top three places in the descending order of content of the contained components, it is considered that the extender powders can be expected to be appropriately contained in the cosmetic, and the entire cosmetic can be effectively used as the paint material.

Further, when the contained components of the extender powders are arranged in the descending order of the content, the extender powders more preferably occupy two of the components in the top three places, and particularly preferably occupy all three of the components in the top three places. Moreover, when the contained components are arranged in the descending order of the content, the extender powders more preferably include the extender powders in the top two places, and particularly preferably include the extender powder in the top one place.

In addition, when the contained components are arranged in the descending order of the content, the extender powders included in the top three places are preferably at least one selected from the group consisting of the mica, the talc, the silica, the synthetic phlogopite (the synthetic fluorofurogopite), and the barium sulfate (the sulfate Ba). These extender powders are bulky powders with a large oil supply amount and are suitable as the paint material, and when used as the paint material, the hiding power, the coloring power, the covering power, the spreadability, the adhesiveness, and the like when spread on the paper can be accommodated. In other words, it is possible to impart a uniformity of adhesion to a surface of the paper, a smoothness of a finished paint film, a visual transparency, and the like.

In the cosmetic to be targeted, when the contained components are arranged in the descending order of the content, the number of the oily components is set to three or less from the top five places. When four or more kinds of the oily components are contained from the top five places, it is considered that a ratio of the oily component to the cosmetic is high, and even if it is mixed with the diluent for the paint material (which will be described in (II) described later) to be used in combination, the diluent is not compatible with the cosmetic and is separated, which becomes unpreferable since the same state as that of the commercially available typical watercolor paint cannot be obtained. In addition, even in a case where the cosmetic and the diluent seemingly can be mixed, a slight separation from the diluent is present, and when the cosmetic is dissolved in the water and smeared on the drawing paper, it may be difficult to use the cosmetic as the paint, which is not preferable. When the contained components in the cosmetic are arranged in the descending order of the content, the number of the oily components is preferably two or less, more preferably one or less, and particularly preferably zero (with no oily components) in the top five places.

In addition, when the contained components are arranged in the descending order of the content, it is preferable that the pearl powder does not appear in the top three places. Since the pearl powder such as a pearlescent pigment (a pearl agent) and a glitter pigment (a lame agent) is formed from powders having a relatively large particle diameter, when the pearl powder appears in the top three places as the coloring material, there is a possibility that a proportion of the pearl powder with respect to the cosmetic increases, and when the pearl powder is formed into the paint, there is a possibility that the pearl powder adversely affects the stretch and a spread on the drawing paper, an over-coating on the drawing paper, the smoothness of the film formed on the drawing paper, and a fixability of the film formed on the drawing paper, and further, there is a possibility that a secondary adhesion is lowered. When the contained components are arranged in the descending order of the content, the pearl powder more preferably does not appear in the top two places, and particularly preferably does not appear in the top place. Further, in regard to the present invention, it is recognized that the “secondary adhesion” refers to an evaluation item rather than a phenomenon, and a performance and a superiority of the evaluation item are described. In other words, the fact that the secondary adhesion becomes low and the secondary adhesion becomes poor indicates that the secondary adhesion is likely to occur when regarded as an event.

The contained components preferably contain at least one selected from the group consisting of the titanium dioxide, the zinc oxide, and the boron nitride. These components are considered to be general-purpose components because they are usually contained in the makeup cosmetic or the like.

In addition to the above components, the components generally used in the cosmetic, for example, desired components generally used in the cosmetic such as drugs, moisturizers, disinfectants, and resins can be added to the cosmetic to be used in the present invention as long as the object and effect of the present invention are not impaired.

In order to use the paint material composed of the cosmetic according to the present invention as a watercolor paint, for example, the paint material is mixed with the diluent for the paint material to be described later (as the mixture) to form the paint. Here, forming into the paint refers to a state similar to that of the commercially available typical watercolor paint (like the paint) (the same applies hereinafter). In addition, after the paint is formed, the paint is dissolved in an appropriate amount of the water and smeared on the drawing paper with a brush in the same way as the commercially available typical watercolor paint (hereinafter, it may be simply referred to as the “typical paint”).

When used in combination with the diluent for the paint material, the paint material composed of such cosmetic according to the present invention has the minimum properties for the paint material, and can be used in the same manner as the typical paint, and the effective use of the cosmetic to be discarded can be achieved. In particular, by using such diluent in combination, it is possible to expect the good characteristics peculiar to the paint material based on the cosmetic in terms of the transparency, the quick-drying property, the smell, and the like of the film when the paint material is stretched and spread on the drawing paper as compared with the typical paint.

Further, when the cosmetic is formed into the paint as in the present invention (hereinafter, it may be referred to as a “cosmetic paint” for convenience), the cosmetic paint may be slightly inferior to the typical paint in the color development (the ease of developing colors). This is because a concentration of the pigment blended in the powdery cosmetic itself produced for the purpose of smearing on the human skin is lower than that of the typical paint.

In addition, depending on a type of the cosmetic to be formed into a paint, there may be a case where a slight decrease in the secondary adhesion (for example, a smeared and dried locus on the drawing paper can be traced with a hand, and can be confirmed whether the locus is attached to the hand or not) is observed as compared with the commercially available typical watercolor paint. The reason is considered to be the fact that the powders such as the extender powders blended in the cosmetic are relatively large as compared with the typical paint having a powder with the particle diameter of substantially the pigment grade.

On the other hand, for example, the film of drawing traces finished with the cosmetic paint has the transparency unique to the cosmetic paint, and may have a touch feeling with the transparency unique to the cosmetic paint, which is not provided in the typical paint. In addition, since the cosmetic paint is originally the cosmetic, it is considered to have a good smell and a quick-drying property and the like are also good by mixing it with the diluent described later.

### (II) Diluent for Paint Material

Next, the diluent for the paint material (hereinafter, it may be simply referred to as the “diluent”) used in combination with the paint material composed of the cosmetic will be described. The diluent is suitable for being used in combination with the paint material or the like composed of the cosmetic according to the present invention described above, and contains the water, the lower alcohol, and the polyvinyl alcohol in which the content thereof is within a specific range. When the cosmetic or the like described above is applied as the paint material (the base material of the paint), such diluent for the paint material is used by being mixed with the cosmetic, thereby playing the role of compensating for the component insufficient as the paint material with respect to the cosmetic.

In other words, since the cosmetic such as the powdery cosmetic is not originally manufactured on an assumption of the paint material, it is natural that there are components insufficient for the typical paint. When the paint material or the like having the above components is used by being dissolved in the water as it is, it is expected that a dispersibility in water, a wettability to water, and the like are insufficient, and even if the paint material or the like is dissolved in water, the adhesiveness to paper, a coatability, the quick-drying property, and the like are insufficient. When the diluent for the paint material contains a specific amount of the above components, such insufficient properties can be supplemented.

The water serves as a solvent for dispersing each component constituting the diluent, and can use generally-used water such as a tap water, a purified water, an ion exchange water. The content of such water may be, for example, a balance (in the diluent, a moiety other than essential components and optional components) excluding the essential components and the optional components in the diluent, but is mostly 49.5 to 89.0 mass %, more preferably 60.5 to 86.0 mass %, and still preferably 66.5 to 83.5 mass % with respect to the entire diluent.

The lower alcohol plays a role as a volatile agent when used in combination with the cosmetic, and can impart a volatility and the quick-drying property. As the lower alcohol, for example, lower alcohols (a monohydric alcohol having 5 or less carbon atoms) such as a methanol (a methyl alcohol), the ethanol (an ethyl alcohol), a propanol (a propyl alcohol), an isopropyl alcohol, a butanol, a propanol can be used, and the ethanol is preferably used from the viewpoint of a versatility, a safety, a compatibility, and the like with the cosmetic.

The content of the lower alcohol is totally 10.0 to 35.0 mass % with respect to the entire diluent. When the content of the lower alcohol is less than 10.0 mass %, the quick-drying property is impaired, the film forming on the paper is delayed, and a color unevenness may occur. On the other hand, when the content is more than 35.0 mass %, the quick-drying property is too strong, and the film is formed without being stretched, which may also cause the color unevenness. Further, when the ethanol is used as the lower alcohol, an excessive ethanol may cause a pigment aggregation. The total content of the lower alcohol is more preferably 12.5 to 30.0 mass %, and is preferably 15.0 to 30.0 mass % with respect to the entire diluent.

The polyvinyl alcohol is useful for the film formation when used in combination with the cosmetic (which becomes a film component), and can impart the covering to the drawing paper, the smoothness to the film, and the like with respect to the film to be formed.

The content of the polyvinyl alcohol is 0.5 to 15.0 mass % with respect to the entire diluent. When the content of the polyvinyl alcohol is less than 0.5 mass %, the film to be formed becomes thin, and the smoothness and the uniformity of the film are lost. On the other hand, when the content exceeds 15.0 mass %, the film becomes thick, the uniformity of the film is damaged, and a finished film may be damaged by a friction. In other words, when the content is small, the film formed on the drawing paper becomes thin, such that irregularities of the powder become conspicuous and the uniformity of the film may be lost, and since the polyvinyl alcohol plays the role of dispersing the pigment, when the content is small, the pigment dispersibility of the paint itself or on the drawing paper at the time of the drawing may be deteriorated, and the color unevenness may occur. On the other hand, when the content is large, the film formed on the drawing paper becomes too thick, such that the uniformity of the film may be lost. The content of the polyvinyl alcohol is more preferably 1.0 to 9.0 mass %, and particularly preferably 1.0 to 3.0 mass % with respect to the entire diluent.

In addition to the essential components described above, the optional components such as a water-soluble film-forming agent and other water-soluble materials, for example, a water-soluble polymer such as the phenoxyethanol, a triethanolamine, a polyhydric alcohol, and a polyacrylic acid (a carbomer), a sodium hydroxide (NaOH), a potassium hydroxide (KOH) (a triethanolamine, the sodium hydroxide, and the potassium hydroxide described above serve as a neutralizing agent for the polyacrylic acid), a nonionic synthetic resin, a nonionic synthetic polymer, an acrylic acid-based water-soluble polymer, a polysaccharide, the surfactant, and the like can be appropriately added to the diluent for the paint material as long as the object and effect of the present invention are not hindered.

Here, it is preferable to solve the problem by using the surfactant for compensating the dispersibility, the wettability (wetting), and the like for the paint material composed of the cosmetic, but since the surfactant is often blended in the cosmetic such as the powdery cosmetic, which is considered to be a target of the paint material, the diluent is mentioned as the optional component in consideration of an influence on the environment and the safety when attached to the human skin when used as the paint. When the optional component is added, the optional component may contain, for example, about 1.0 mass % or less with respect to the entire diluent, but is not limited thereto.

The diluent for the paint material can be prepared by mixing and stirring the essential components and optional components mentioned above (the essential components and the like). As an example, each component may be added to the water to be the solvent, and the essential components and the like may be dispersed in the water by mixing and stirring.

When the diluent for the paint material according to the present invention described above is used in combination with the paint material composed of the cosmetic, the diluent for the paint material can play the role of compensating for the component insufficient as the paint material with respect to the cosmetic for the paint material.

### (III) Paint and Paint Set

The paint according to the present invention includes the mixture obtained by mixing the paint material composed of the cosmetic described in (I) and the diluent for the paint material described in (II), and similarly, the paint set according to the present invention includes the paint material composed of the cosmetic described in (I) and the diluent for the paint material described in (II). As the paint set, one kind of each of the paint materials and the diluent that compose of the cosmetic may be used, or two or more kinds thereof may be used.

The paint material composed of the cosmetic is diluted with the diluent for the paint material to form the mixture, such that the state similar to that of the commercially available typical watercolor paint (like the typical paint) is obtained (which is the paint of the present invention). A blending ratio of the paint material and the diluent is not particularly limited and can be appropriately determined according to the type of the cosmetic, a blending percentage of the components constituting the diluent, a color finally obtained by a user, and the like; but mostly, it is preferable to mix the diluent at 0.5 to 2 and particularly preferable to mix the diluent at 0.7 to 1.5 with respect to cosmetic 1 as a mass ratio, yet this range is not particularly limited.

The paint material diluted with the diluent (the paint as the mixture) is dissolved in the water on a pallet or the like and used, for example, as in the commercially available typical watercolor paint. The amount of the water to be used may be mostly, as a mass ratio, used in such a manner that the water is mixed and dissolved as 0.5 to 3 with respect to 1 of the diluted paint material (a cosmetic formed into a paint having the mixture of the cosmetic and the diluent), but is not limited to such a mixing ratio, and may be appropriately determined according to user's preference or the like.

The paint and the paint set according to the present invention explained above include the paint material composed of the cosmetic according to the present invention and the diluent for the paint material according to the present invention, and can be provided as the paint that can be used in the same manner as the typical paint and as the paint set in which the paint material and a dedicated diluent are included.

### (V) Embodiment Variants

In addition, the aspects described above illustrate one aspect of the present invention, the present invention is not limited to the embodiments described above, and it goes without saying that modifications and improvements having the configuration of the present invention and within a scope of achieving the object and the effect are included in the content of the present invention. Further, a specific structure, a shape, and the like in carrying out the present invention may be other structures, shapes, and the like as long as the object and effect of the present invention can be achieved. The present invention is not limited to the embodiments described above, and the modifications and the improvements within the scope of achieving the object of the present invention are included in the present invention.

For example, in the embodiments described above, a certain type of cosmetic is exemplified as the type of cosmetic that can be used as the paint material, but the cosmetic that can be used as the paint material is not limited thereto, and an application of the cosmetic such as the powdery cosmetic other than those exemplified is not limited. Similarly, in the above embodiments, specific examples of each component are given but are merely examples, and there is no problem even if the component not given is blended in the paint material composed of the cosmetic.

In addition, the specific structure, shape, and the like at the time of carrying out the present invention may be other structures and the like as long as the object of the present invention can be achieved.

### EXAMPLES

Hereinafter, the present invention will be described in more detail based on test examples and the like, but the present invention is not limited thereto.

16 kinds of commercially available cosmetics A to P having compositions shown in Tables 1 to 9 were prepared (Table 1 shows product names and types, and Tables 2 to 9 show all component information such as composition). The cosmetics A to P have the color development suitable for use of the coloring material as the component for the paint material (an evaluation is also performed in Test Example 2(4) described later).

Further, the cosmetics A to P contain the extender powders, the oily components, and the like, in which the cosmetics A to G and the cosmetics M to P are respectively classified into the eye shadows, the cosmetic H is classified into the blushers, the cosmetics I to K are classified into the face powders, and the cosmetic L is classified into the foundations. In addition, the cosmetic A, the cosmetic B, the cosmetic D, and the cosmetic H to the cosmetic P are generally distributed and sold as the so-called powdery cosmetic.

(Product Names and Manufacturers/Distributors)

TABLE 1

| Product names of cosmetics | Manufacturers/Distributors |
|---|---|
| Cosmetic A: It girl eye shadow kuchibue | ITG Eye Shadow C |
| Cosmetic B: Rimmel prism powder eye color 010 (red) | Cosme Labo Co., Ltd. |
| Cosmetic C: Rimmel prism cream eye color 011 (yellow) | Cosme Labo Co., Ltd. |
| Cosmetic D: Noah eye color S(N) 20 (blue) | KOSÉ Corporation |
| Cosmetic E: Canmake jewel star eyes | IDA Laboratories Co., Ltd. |
| Cosmetic F: Kancore cream eye shadow khaki | Erato Co., Ltd. |
| Cosmetic G: UR GLAM sexy and healthy makeup airy eye color PK-2 | Do-Best, Inc. |
| Cosmetic H: UG daily cheek blush CO-1 | Do-Best, Inc. |
| Cosmetic I: Espoleur pressed powder 2D4G4 lavender pearl (face powder) | Daiso Industries Co., Ltd. |
| Cosmetic J: UR GRAM sexy and healthy makeup oil control powder (face powder) | Do-Best, Inc. |
| Cosmetic K: Kanebo Media pressed powder AA | Kanebo Cosmetics Inc. |
| Cosmetic L: Cezanne ultra-cover UV foundation II #3 | Cezanne Cosmetics Co., Ltd. |
| Cosmetic M: BW milico eye shadow (earth green) | Star-lab Cosmetics Co., Ltd. |
| Cosmetic N: Palgantong sparkling eye shadow SE115 gold | Dodo Japan Co., Ltd. |
| Cosmetic O: Kate the eye color 011 pearl orange beige eye shadow | Kanebo Cosmetics Inc. |
| Cosmetic P: Kate the eye color 032 matte red eye shadow | Kanebo Cosmetics Inc. |

(All Component Information Such as the Composition: The Cosmetic a and the Cosmetic B)

TABLE 2

| | | B: Rimmel prism powder | |
|---|---|---|---|
| A: It girl eye shadow kuchibue | Classification | eye color 010 (red) | Classification |
| Mica | Extender powder | Synthetic phlogopite | Extender powder |
| Talc | Extender powder | Triethylhexanoin | Oily component |
| | | | (ester oil) |
| Corn starch | Extender powder | Tritridecyl trimellitate | Oily component |
| | | | (ester oil) |
| Tritridecyl trimellitate | Oily component | Talc | Extender powder |
| | (ester oil) | | |
| Silica | Extender powder | Dimethicone | Oily component |
| | | | (silicone oil) |
| Mg stearate | Extender powder | Petrolatum | Oily component |
| | | | (hydrocarbon oil) |
| Ethylhexyl palmitate | Oily component | Methyl methacrylate | Extender powder |
| | (ester oil) | crosspolymer | |
| Phenoxyethanol | Aqueous preservative | Sorbitan | Surfactant |
| | | sesquiisostearate | |
| Polybutene | Oily component | Isododecane | Oily component |
| | (hydrocarbon oil) | | (organic solvent) |
| Caprylyl glycol | Aqueous preservative | Tin oxide | Coloring material |
| | | | (pearl powder) |
| Mineral oil | Oily component | Al hydroxide | Surface treatment agent |
| | (hydrocarbon oil) | | |
| (Ethylene/propylene/styrene) | Oily component | Ultramarine | Coloring material |
| copolymer | (oil thickener) | | (inorganic pigment) |
| (Butylene/ethylene/styrene) | Oily component | Mica | Extender powder |
| copolymer | (oil thickener) | | |
| Subsequent (+/−) | | Titanium dioxide | Coloring material |
| | | | (white pigment and |
| | | | pearl powder) |
| Yellow 4 | Coloring material | Zinc oxide | Coloring material |
| | (organic pigment) | | (white pigment) and |
| | | | powdery preservative |
| Yellow 5 | Coloring material | Iron oxide | Coloring material |
| | (organic pigment) | | (inorganic pigment) |
| Red 201 | Coloring material | Red 202 | Coloring material |
| | (organic pigment) | | (organic pigment) |
| Red 202 | Coloring material | Red 226 | Coloring material |
| | (organic pigment) | | (organic pigment) |
| Carmine | Coloring material | | |
| | (natural pigment) | | |
| Titanium dioxide | Coloring material | | |
| | (white pigment and | | |
| | pearl powder) | | |
| Iron oxide | Coloring material | | |
| | (inorganic pigment) | | |
| Tin oxide | Coloring material | | |
| | (pearl powder) | | |
| Orange 205 | Coloring material | | |
| | (organic pigment) | | |

(All Component Information Such as the Composition: The Cosmetic C and the Cosmetic D)

TABLE 3

| C: Rimmel prism cream | | D: Noah eye color | |
|---|---|---|---|
| eye color 011 (yellow) | Classification | S(N) 20 (blue) | Classification |
| Triethylhexanoin | Oily component | Dimethicone | Oily component |
| | (ester oil) | | (silicone oil) |
| PG dicaprate | Oily component | Diisostearyl malate | Oily component |
| | (ester oil) | | (ester oil) |
| Polymethylsilsesquioxane | Extender powder | Talc | Extender powder |
| Polyglyceryl-2 | Oily component | Cholesteryl | Oily component |
| triisostearate | (ester oil)/surfactant | hydroxystearate | (ester oil) |
| Dimethicone | Oily component | Chamomile extract | Cosmetic agent |
| | (silicone oil) | | |
| (Vinyl dimethicone/ | Extender powder | Tocopherol | Antioxidant |
| methicone silsesquioxane) | | | |
| crosspolymer | | | |
| Borosilicate (Ca/Al) | Coloring material | Sorbitan stearate | Surfactant |
| | (pearl powder) | | |
| (Ethylene/propylene) | Oily component | Octyldodecyl myristate | Oily component |
| copolymer | (oil thickener) | | (ester oil) |
| Hexa(hydroxystearic | Oily component | Methyl paraben | Preservative |
| acid/stearic acid/rosin | (ester oil) | | |
| acid) dipentaerythrityl | | | |
| Synthetic phlogopite | Extender powder | Subsequent (+/−) | |
| Silica | Extender powder | (PET/polymethyl | Coloring material |
| | | methacrylate) laminate | (pearl powder) |
| Candelilla wax | Oily component (wax) | Ultramarine | Coloring material |
| | | | (inorganic pigment) |
| BHT | Antioxidant | Ferric ferrocyanide | Coloring material |
| | | | (inorganic pigment) |
| Dimethylsilylated silica | Extender powder | Mica | Extender powder |
| Hydrogen dimethicone | Oily component | Titanium dioxide | Coloring material |
| | (silicone oil) | | (white pigment and |
| | | | pearl powder) |
| Perfluorohexylethyltriethoxysilane | Surface treatment agent | Iron oxide | Coloring material |
| | | | (inorganic pigment) |
| Paraffin | Oily component | Al hydroxide | Surface treatment agent |
| | (hydrocarbon oil) | | |
| Microcrystalline wax | Oily component (wax) | Sulfate Ba | Extender powder |
| Tin oxide | Coloring material | Red 226 | Coloring material |
| | (pearl powder) | | (organic pigment) |
| Methyl paraben | Preservative | | |
| Ultramarine | Coloring material | | |
| | (inorganic pigment) | | |
| Mica | Extender powder | | |
| Titanium dioxide | Coloring material | | |
| | (white pigment and | | |
| | pearl powder) | | |
| Iron oxide | Coloring material | | |
| | (inorganic pigment) | | |
| Yellow 4 | Coloring material | | |
| | (organic pigment) | | |

(All Component Information Such as the Composition: The Cosmetic E and the Cosmetic F)

TABLE 4

| E: Canmake jewel star eyes | Classification | F: Kancore cream eye shadow khaki | Classification |
|---|---|---|---|
| Isotridecyl | Oily component | Dimethicone | Oily component |
| isonanoate | (ester oil) | | (silicone oil) |
| Squalane | Oily component | Glyceryl tri-(caprylic | Oily component |
| | (hydrocarbon oil) | acid/capric acid) | (ester oil) |
| Dimethicone | Oily component | Polybutene | Oily component |
| | (silicone oil) | | (hydrocarbon oil) |
| Dextrin palmitate | Oily component | Octyldodecanol | Oily component |
| | (oil thickener) | | (higher alcohol) |
| Methyl methacrylate | Extender powder | Isononyl isononanoate | Oily component |
| crosspolymer | | | (ester oil) |
| (Vinyl dimethicone/ | Extender powder | Microcrystalline wax | Oily component (wax) |
| methicone silsesquioxane) | | | |
| crosspolymer | | | |
| Sorbitan | Surfactant | Isododecane | Oily component |
| sesquiisostearate | | | (organic solvent) |
| Polyglyceryl-2 | Oily component | Polyethylene | Extender powder/Oily |
| triisostearate | (ester oil)/surfactant | | component (wax) |
| (Dimethicone/vinyl | Extender powder | Silica | Extender powder |
| dimethicone) | | | |
| crosspolymer | | | |
| DimethylsilylatedSilica | Extender powder | Ceresin | Oily component (wax) |
| Tocopherol | Antioxidant | Ozokerite | Oily component (wax) |
| Di(phytosteryl/ | Ceramide (cosmetic | Hydrogenated vegetable oil | Oily component |
| octyldodecyl) lauroyl | agent) | | (vegetable oil) |
| glutamate | | | |
| Borosilicate (Ca/Al) | Coloring material | Disteardimonium hectorite | Extender powder |
| | (pearl powder) | | |
| Titanium dioxide | Coloring material | Beeswax | Oily component (wax) |
| | (white pigment and | | |
| | pearl powder) | | |
| Mica | Extender powder | Phenoxyethanol | Aqueous preservative |
| Iron oxide | Coloring material | Hydrogenated palm oil fatty acid | Oily component (wax) |
| | (inorganic pigment) | | |
| Silica | Extender powder | Stearyl stearate | Oily component (wax) |
| Hydrogen Dimethicone | Oily component | Caprylyl glycol | Preservative |
| | (silicone oil) | | |
| Alumina | Extender powder | Lecithin | Cosmetic agent |
| | | Polyhydroxystearic acid | Surfactant |
| | | Isopropyl myristate | Oily component |
| | | | (ester oil) |
| | | Ethylhexyl palmitate | Oily component |
| | | | (ester oil) |
| | | Isostearic acid | Surface treatment agent |
| | | Polyglyceryl polyricinoleate-3 | Surfactant |
| | | BHT | Antioxidant |
| | | Subsequent (+/−) | |
| | | Mica | Extender powder |
| | | Iron oxide | Coloring material |
| | | | (inorganic pigment) |
| | | Titanium dioxide | Coloring material |
| | | | (white pigment and |
| | | | pearl powder) |
| | | Synthetic fluorofurogopite | Extender powder |
| | | (synthetic phlogopite) | |
| | | Borosilicate (Ca/Al) | Coloring material |
| | | | (pearl powder) |
| | | Tin oxide | Coloring material |
| | | | (pearl powder) |

(All Component Information Such as the Composition: The Cosmetic G and the Cosmetic H)

TABLE 5

| G: UR GLAM sexy and healthy | | | |
|---|---|---|---|
| makeup airy eye color PK-2 | Classification | H: UG daily cheek blush C0-1 | Classification |
| Ethylhexyl palmitate | Oily component | Mica | Extender powder |
| | (ester oil) | | |
| Silica | Extender powder | Talc | Extender powder |
| Cyclopentasiloxane | Oily component | Corn starch | Extender powder |
| | (silicone oil) | | |
| Glyceryl tri-(caprylic | Oily component | Silica | Extender powder |
| acid/capric acid) | (ester oil) | | |
| Bisdiglyceryl polyacyl | Oily component | Tritridecyl trimellitate | Oily component |
| adipate-2 | (ester oil) | | (ester oil) |
| Isododecane | Oily component | Silicic acid (Na/K/Al) | Extender powder |
| | (organic solvent) | | |
| Microcrystalline wax | Oily component (wax) | Dimethicone | Oily component |
| | | | (silicone oil) |
| (Dimethicone/vinyl | Extender powder | Mg stearate | Extender powder |
| dimethicone) crosspolymer | | | |
| Hydroxystearic acid | Oily component | Ethylhexyl palmitate | Oily component |
| | (higher fatty acid) | | (ester oil) |
| Paraffin | Oily component | Polybutene | Oily component |
| | (hydrocarbon oil) | | (hydrocarbon oil) |
| Stearic acid | Surface treatment agent | Mineral oil | Oily component |
| | | | (hydrocarbon oil) |
| Hydrogenated tri-fatty | Oily component | (Ethylene/propylene/styrene) | Oily component |
| acid (C12-18) glyceryl | (ester oil) | copolymer | (oil thickener) |
| Beeswax | Oily component (wax) | (Butylene/ethylene/styrene) | Oily component |
| | | copolymer | (oil thickener) |
| Palmitic acid | Surface treatment agent | Phenoxyethanol | Aqueous preservative |
| Phenoxyethanol | Aqueous preservative | Methyl paraben | preservative |
| Methyl paraben | Preservative | Ethyl paraben | preservative |
| Ethyl paraben | Preservative | Iron oxide | Coloring material |
| | | | (inorganic pigment) |
| Mica | Extender powder | Titanium dioxide | Coloring material |
| | | | (white pigment and |
| | | | pearl powder) |
| Borosilicate (Ca/Al) | Coloring material | Tin oxide | Coloring material |
| | (pearl powder) | | (pearl powder) |
| Titanium dioxide | Coloring material | Borosilicate (Ca/Na) | Coloring material |
| | (white pigment and | | (pearl powder) |
| | pearl powder) | | |
| Tin oxide | Coloring material | Red 201 | Coloring material |
| | (pearl powder) | | (organic pigment) |
| Al hydroxide | Surface treatment agent | Red 202 | Coloring material |
| | | | (organic pigment) |
| Iron oxide | Coloring material | Yellow 4 | Coloring material |
| | (inorganic pigment) | | (organic pigment) |
| Red 201 | Coloring material | | |
| | (organic pigment) | | |
| Red 226 | Coloring material | | |
| | (organic pigment) | | |
| Yellow 4 | Coloring material | | |
| | (organic pigment) | | |

(All Component Information Such as the Composition: The Cosmetic I and the Cosmetic J)

TABLE 6

| I: Espoleur pressed powder | | J: UR GRAM sexy and | |
|---|---|---|---|
| 2D4G4 lavender pearl | | healthy makeup oil control | |
| (face powder) | Classification | powder (face powder) | Classification |
| Talc | Extender powder | Talc | Extender powder |
| Aluminum corn starch | Extender powder | Mica | Extender powder |
| octenylsuccinate | | | |
| Triethylhexanoin | Oily component | Silica | Extender powder |
| | (ester oil) | | |
| Titanium dioxide | Coloring material | Methyl methacrylate | Extender powder |
| | (white pigment and | crosspolymer | |
| | pearl powder) | | |
| Zinc oxide | Coloring material | Polyethylene | Extender powder |
| | (white pigment) and | | |
| | powdery preservative | | |
| (Diphenyl dimethicone/ | Extender powder | Tritridecyl trimellitate | Oily component |
| vinyl diphenyl | | | (ester oil) |
| dimethicone/silsesquioxane) | | | |
| crosspolymer | | | |
| Strawberry fruit extract | Cosmetic agent | Ethylhexyl palmitate | Oily component |
| | | | (ester oil) |
| Lavender flower extract | Cosmetic agent | Stearic acid Mg | Extender powder |
| Maggot root bark extract | Cosmetic agent | Dimethicone | Oily component |
| | | | (silicone oil) |
| Water | Aqueous component | Triethoxycaprylylsilane | Surface treatment agent |
| BG | Aqueous component/ | Phenoxyethanol | Aqueous preservative |
| | preservative | | |
| Hydrogenated starch | Extender powder | Iron oxide | Coloring material |
| hydrolysate | | | (inorganic pigment) |
| Hexa(hydroxystearic | Oily component | | |
| acid/stearic acid/rosin | (ester oil) | | |
| acid) dipentaerythrityl | | | |
| Stearic acid Mg | Extender powder | | |
| Silica | Extender powder | | |
| Al hydroxide | Surface treatment agent | | |
| Methyl paraben | Preservative | | |
| Hydrogen Dimethicone | Oily component | | |
| | (silicone oil) | | |
| Methicone | Oily component | | |
| | (silicone oil) | | |
| Subsequent (+/−) | | | |
| Mica | Extender powder | | |
| Manganese violet | Coloring material | | |
| | (inorganic pigment) | | |

(All Component Information Such as the Composition: The Cosmetic K and the Cosmetic L)

TABLE 7

| K: Kanebo Media | | L: Cezanne ultra-cover | |
|---|---|---|---|
| pressed powder AA | Classification | UV foundation II #3 | Classification |
| Talc | Extender powder | Mica | Extender powder |
| Sulfate Ba | Extender powder | Titanium dioxide | Coloring material |
| | | | (white pigment and |
| | | | pearl powder) |
| Mica | Extender powder | Silica | Extender powder |
| Polymethyl | Extender powder | Zinc oxide | Coloring material |
| methacrylate | | | (white pigment) and |
| | | | powdery preservative |
| Dimethicone | Oily component | Zinc myristate | Extender powder |
| | (silicone oil) | | |
| Isononyl isononanoate | Oily component | Al hydroxide | Surface treatment agent |
| | (ester oil) | | |
| Ethylhexyl | Ultraviolet absorber | (Dimethicone/vinyl | Extender powder |
| methoxycinnamate | | dimethicone) crosspolymer | |
| Lauroyllysine | Extender | Hydroxyapatite | Extender powder |
| | powder/surface | | |
| | treatment agent | | |
| Isostearic | Oily component | Diphenylsiloxyphenyl | Oily component |
| acidhydrogenated | (ester oil) | trimethicone | (silicone oil) |
| castor oil | | | |
| Octyldodecanol | Oily component | Stearic acid | Surface treatment agent |
| | (higher alcohol) | | |
| Butylene glycol (BG) | Aqueous component/ | Hydrogen dimethicone | Oily component |
| | preservative | | (silicone oil) |
| Zinc myristate | Extender powder | Squalane | Oily component |
| | | | (hydrocarbon oil) |
| Silica | Extender powder | (Vinyl dimethicone/methicone | Extender powder |
| | | silsesquioxane) crosspolymer | |
| Water-soluble collagen | Cosmetic agent | Lauroyllysine | Extender powder/surface |
| | | | treatment agent |
| Hydrolyzed conchiolin | Cosmetic agent | Diisostearyl malate | Oily component |
| | | | (ester oil) |
| Methyl paraben | Preservative | Hexa(hydroxystearic | Oily component |
| | | acid/stearic acid/rosin | (ester oil) |
| | | acid) dipentaerythrityl | |
| Ethyl paraben | Preservative | Methicone | Oily component |
| | | | (silicone oil) |
| Subsequent (+/−) | | Mg myristate | Extender powder |
| Titanium dioxide | Coloring material | Chitosan | Cosmetic agent |
| | (white pigment and | | |
| | pearl powder) | | |
| Zinc oxide | Coloring material | Aluminum starch | Extender powder |
| | (white pigment) and | Octenylsuccinate | |
| | powdery preservative | | |
| Iron oxide | Coloring material | Sulfate Ba | Extender powder |
| | (inorganic pigment) | | |
| Al hydroxide | Surface treatment | Trimethylsiloxysilicic acid | Film-forming agent |
| | agent | | |
| | | Tocopherol | Antioxidant |
| | | BG | Aqueous component/ |
| | | | preservative |
| | | Water | Aqueous component |
| | | Lactobacilli/hibiscus flower | Cosmetic agent |
| | | fermentation liquid | |
| | | Rhodomyrtus tomentosa | Cosmetic agent |
| | | fruit extract | |
| | | Methyl paraben | Preservative |
| | | Propyl paraben | Preservative |
| | | Talc | Extender powder |
| | | Dimethicone | Oily component |
| | | | (silicone oil) |
| | | Iron oxide | Coloring material |
| | | | (inorganic pigment) |

(All Component Information Such as the Composition: The Cosmetic M and the Cosmetic N)

TABLE 8

| M: BW milico eye shadow | | N: Palgantong sparkling eye | |
|---|---|---|---|
| (earth green) | Classification | shadow SE115 gold | Classification |
| Talc | Extender powder | Borosilicate (Ca/Al) | Coloring material |
| | | | (pearl powder) |
| Mica | Extender powder | Talc | Extender powder |
| Stearic acid Mg | Extender powder | Synthetic fluorofurogopite | Extender powder |
| | | (synthetic phlogopite) | |
| Dimethicone | Oily component | Neopentyl glycol | Oily component |
| | (silicone oil) | diethylhexanoate | (ester oil) |
| Ethylhexyl palmitate | Oily component | Phenyl trimethicone | Oily component |
| | (ester oil) | | (silicone oil) |
| Polybutene | Oily component | Polypropylene | Extender powder |
| | (hydrocarbon oil) | | |
| Synthetic wax | Oily component (wax) | Diisostearyl malate | Oily component |
| | | | (ester oil) |
| Mineral oil | Oily component | Hexa(hydroxystearic acid/ | Oily component |
| | (hydrocarbon oil) | stearic acid/rosin acid) | (ester oil) |
| | | dipentaerythrityl | |
| T-butyl | Ultraviolet absorber | Octyldodecyl | Oily component |
| methoxydibenzoylmethane | | stearoyloxystearate | (ester oil) |
| Ethylhexyl | Ultraviolet absorber | Isostearyl neopentanoate | Oily component |
| methoxycinnamate | | | (ester oil) |
| Diethylhexyl syringlidene | Antioxidant | Camellia seed oil | Cosmetic agent |
| malonate | | | |
| Ethylhexylglycerin | Preservative | Mg myristate | Extender powder |
| Tri(caprylic acid/capric | Emollient component | Stearic acid Mg | Extender powder |
| acid)glyceryl | | | |
| Phenoxyethanol | Aqueous preservative | Methyl methacrylate | Extender powder |
| | | crosspolymer | |
| BHT | Antioxidant | Polybutene | Oily component |
| | | | (hydrocarbon oil) |
| Subsequent (+/−) | | Propanediol | Preservative |
| Titanium dioxide | Coloring material | Polytetrafluoroethylene | Extender powder |
| | (white pigment and | | |
| | pearl powder) | | |
| Tin oxide | Coloring material | Triethoxycaprylylsilane | Surface treatment agent |
| | (pearl powder) | | |
| Iron oxide | Coloring material | Tin oxide | Coloring material |
| | (inorganic pigment) | | (pearl powder) |
| Ultramarine | Coloring material | Dimethicone | Oily component |
| | (inorganic pigment) | | (silicone oil) |
| Ferric ferrocyanide | Coloring material | Tocopherol acetate | Antioxidant |
| | (inorganic pigment) | | |
| Synthetic fluorofurogopite | Extender powder | Sodium dehydroacetate | Preservative |
| Borosilicate (Ca/Al) | Coloring material | Mica | Extender powder |
| | (pearl powder) | | |
| Sulfate Ba | Extender powder | Titanium dioxide | Coloring material |
| | | | (white pigment and |
| | | | pearl powder) |
| Al hydroxide | surface treatment agent | Iron oxide | Coloring material |
| | | | (inorganic pigment) |
| Red 201 | Coloring material | Silver | Preservative |
| | (organic pigment) | | |
| Red 202 | Coloring material | | |
| | (organic pigment) | | |
| Yellow 4 | Coloring material | | |
| | (organic pigment) | | |
| Blue 1 | Coloring material | | |
| | (organic pigment) | | |

(All Component Information Such as the Composition: The Cosmetic O and the Cosmetic P)

TABLE 9

| O: Kate the eye color | | | |
|---|---|---|---|
| 011 pearl orange beige | | P: Kate the eye color | |
| eye shadow | | 032 matte red eye shadow | Classification |
| Talc | Extender powder | Talc | Extender powder |
| Mica | Extender powder | Mica | Extender powder |
| Synthetic fluorofurogopite | Extender powder | Synthetic | Extender powder |
| (synthetic phlogopite) | | fluorofurogopite(synthetic | |
| | | phlogopite) | |
| Diisostearyl malate | Oily component | Diisostearyl malate | Oily component |
| | (ester oil) | | (ester oil) |
| Squalane | Oily component | Squalane | Oily component |
| | (hydrocarbon oil) | | (hydrocarbon oil) |
| Dimethicone | Oily component | Dimethicone | Oily component |
| | (silicone oil) | | (silicone oil) |
| Petrolatum | Oily component | Petrolatum | Oily component |
| | (hydrocarbon oil) | | (hydrocarbon oil) |
| Methyl methacrylate | Extender powder | Methyl methacrylate | Extender powder |
| crosspolymer | | crosspolymer | |
| Sorbitan sesquiisostearate | Surfactant | Sorbitan sesquiisostearate | Surfactant |
| Aluminum distearate | Extender powder | Aluminum distearate | Extender powder |
| Tocopherol | Antioxidant | Tocopherol | Antioxidant |
| Ethyl paraben | Preservative | Ethyl paraben | Preservative |
| Methyl paraben | Preservative | Methyl paraben | Preservative |
| Na dehydroacetate | Preservative | Na dehydroacetate | Preservative |
| Subsequent (+/−) | | Subsequent (+/−) | |
| Ultramarine | Coloring material | Ultramarine | Coloring material |
| | (inorganic pigment) | | (inorganic pigment) |
| Silica | Extender powder | Silica | Extender powder |
| Titanium dioxide | Coloring material | Titanium dioxide | Coloring material |
| | (white pigment and | | (white pigment and |
| | pearl powder) | | pearl powder) |
| Iron oxide | Coloring material | Iron oxide | Coloring material |
| | (inorganic pigment) | | (inorganic pigment) |
| Tin oxide | Coloring material | Tin oxide | Coloring material |
| | (pearl powder) | | (pearl powder) |
| Borosilicate (Ca/Al) | Coloring material | Borosilicate (Ca/Al) | Coloring material |
| | (pearl powder) | | (pearl powder) |
| Red 202 | Coloring material | Red 202 | Coloring material |
| | (organic pigment) | | (organic pigment) |
| Red 226 | Coloring material | Red 226 | Coloring material |
| | (organic pigment) | | (organic pigment) |

Here, the compositions of Table 2 and the like are arranged in the descending order of the component amount with respect to the entire cosmetic. Further, in the compositions of Table 2 and the like, the display of “+/−” is a display used in a series product with many color differences such as the cosmetic for makeup, and indicates that the component (the coloring material or the like) displayed thereafter is used in the series product. Accordingly, the component that is not necessarily blended may be displayed in the cosmetic having a described product name (however, hereinafter at least one coloring material represented by the “+/−” is used).

Then, for the 16 kinds of the cosmetics, various properties of the paint material were compared and evaluated through the method described in [Test Example 2] using the diluent for the paint material selected in [Test Example 1] below.

### Test Example 1

Evaluation of the Diluent for the Paint Material:

The diluent for the paint material (the diluent) obtained through the following method was evaluated for a suitability as the diluent for the paint material using the cosmetic A as a control.

(Production of the Diluent for the Paint Material)

As the components of the diluent for the paint material, the water, the ethanol as the lower alcohol, the polyvinyl alcohol, and the phenoxyethanol were set to the contents (mass % with respect to the entire diluent) shown in Table 10, and other components were added to the water as the solvent, mixed, and stirred to be dispersed in the water, thereby producing diluents 1 to 3.

(Composition of the Diluent)

TABLE 10
[mass %]

| | Diluent 1 | Diluent 2 | Diluent 3 |
|---|---|---|---|
| Water | 83.5 | 73.5 | 71.5 |
| Ethanol | 15.0 | 25.0 | 25.0 |
| Polyvinyl alcohol | 1.0 | 1.0 | 3.0 |
| Phenoxyethanol | 0.5 | 0.5 | 0.5 |

The diluent 1 to the diluent 3 thus obtained were formed into the paint (to be in the same state as the commercially available typical watercolor paint (like the typical paint)) through the method below using the cosmetic A listed in Tables 1 and 2, and then an evaluation sample was prepared, and the suitability as the diluent for the paint material was evaluated through the method below.

(Forming into the Paint and Preparing the Evaluation Sample)

The cosmetic (3 g) and the diluent (3 g) were kneaded with a spatula for five minutes to form the mixture, and the cosmetic was formed into the paint. Then, 0.2 g of the cosmetic formed into the paint and 0.2 g of the water (the tap water) were dissolved on the pallet to prepare the evaluation sample.

(Evaluation Method)

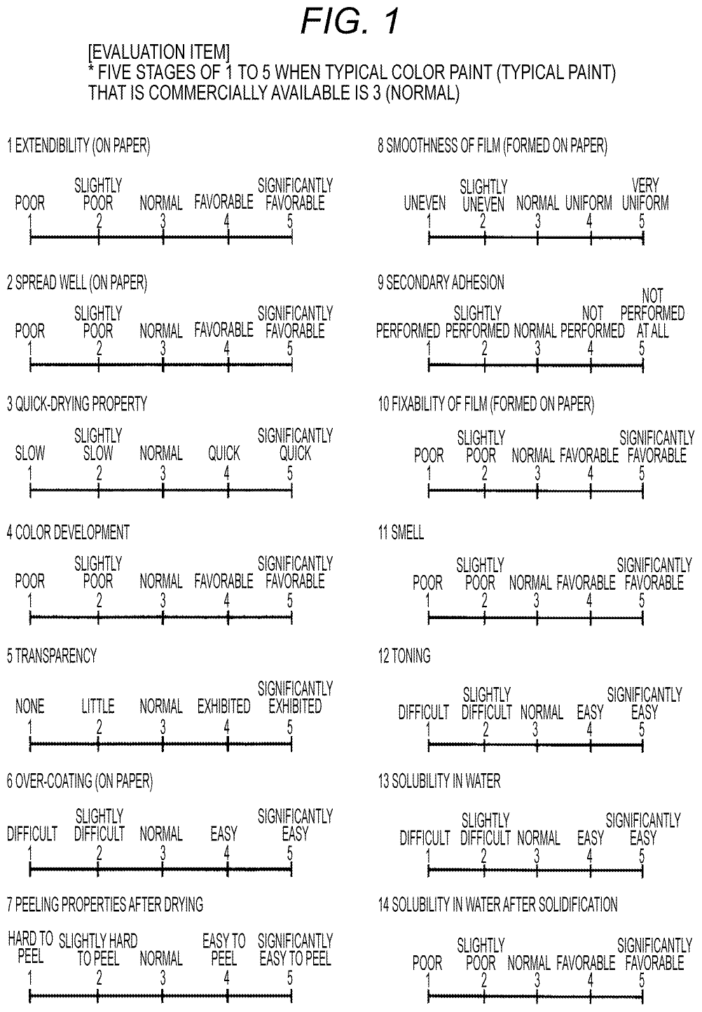

The evaluation samples were evaluated by 12 panelists according to the criteria shown in FIG. 1 (when three commercially available typical watercolor paints were used, the number of points is 5, 4, 3, 2, and 1, which are the five levels, with 5 being the best and 1 being the worst) using five items of (1) extendibility (on paper), (2) spread well (on paper), (3) quick-drying property, (5) transparency, and (8) smoothness of film (formed on paper) among the 14 evaluation items of (1) to (14) below, and an average score (an average value) of the 12 panelists was calculated. FIG. 1 is a diagram showing the evaluation criteria used in Test Example 1 and Test Example 2, and these criteria are also used in Test Example 2 described later.

Then, the calculated average scores were compared and evaluated using the evaluation criteria below. The results are shown in Table 11 (in Table 11 and Table 12 described later, the average scores of the results and evaluations of “⊙,” “◯,” “Δ,” and “x” are listed below). Additionally, in FIG. 1 and the contents below, the “drawing paper” to be smeared may be simply described as the “paper.” Further, the average values in Table 11 and Table 12 described later are rounded off to the third decimal place in some cases.

(Evaluation Criteria)

Evaluation: Content

⊙: the average score of 3.5 points or more

∘: the average score of 2.5 points or more and less than 3.5 points

Δ: the average score of 1.5 points or more and less than 2.5 points

x: the average score of less than 1.5 points

(Evaluation Items)

(1) Extendibility (on Paper):

The ease of stretching and spreading on the paper when painted on the drawing paper with the brush was confirmed.

(2) Spread Well (on Paper):

It was confirmed whether the smeared film could be smeared on the paper in the same manner as in the case of the typical paint without blurring.

(3) Quick-Drying Property:

The drying speed was confirmed.

(4) Color Development:

The ease of developing the color was confirmed.

(5) Transparency:

The degree to which the drawing paper was seen through was confirmed.

(6) Over-Coating (on Paper):

Immediately after smearing, the smearing was performed again from above, and whether the first smearing was twisted or taken to the second smearing was confirmed.

(7) Peeling Properties after Drying:

After putting the paint on the pallet and solidifying it for several days, whether the paint would fall off or peel off from the pallet was confirmed.

(8) Smoothness of Film (Formed on Paper):

The drawing paper was smeared and the dried locus was traced by hand, and whether the drawing paper was rough or uneven was confirmed.

(9) Secondary Adhesion:

The smeared and dried locus on the drawing paper was traced by hand, and whether the smeared and dried locus adheres to the hand was confirmed.

(10) Fixability of Film (Formed on Paper):

After smearing and drying, whether the film was twisted when being traced by hand was confirmed.

(11) Smell:

Whether there was the good smell with respect to the unique smell of the typical paint was confirmed.

(12) Toning:

Whether the color was as expected when other cosmetic paints were mixed on the pallet was confirmed (for example, when red and blue are mixed, the color is expected to be purple, which needs to be confirmed).

(13) Solubility in Water:

Whether the paint was easy to mix when mixing with the water on the pallet was confirmed.

(14) Solubility in Water after Solidification:

The paint was placed on the pallet in the same manner as (7), dried and solidified, and then whether the paint could be redissolved in water or not was confirmed.

(Results)

TABLE 11

| | Evaluation items (point: average score) | | | | |
|---|---|---|---|---|---|
| | (1) | (2) | (3) | (5) | (8) |
| Cosmetic A + Diluent 1 | 3.42 ◯ | 3.08 ◯ | 4.58 ⊙ | 5 ⊙ | 3.33 ◯ |
| Cosmetic A + Diluent 2 | 3.25 ◯ | 3.17 ◯ | 4.75 ⊙ | 5 ⊙ | 3.33 ◯ |
| Cosmetic A + Diluent 3 | 3 ◯ | 3.5 ⊙ | 4.92 ⊙ | 5 ⊙ | 3.5 ⊙ |

As shown in Table 11, according to the content of the lower alcohol (the ethanol) considered to be the volatile agent and the content of the polyvinyl alcohol (PVA) considered to be the film component, the results showed that the evaluation scores of (2) spread well (on paper), (3) quick-drying property, and (8) smoothness of film were increased (the diluent 3 is the highest). Further, regarding (5) transparency, all the three diluents gave the average score of 5 points (the full score).

On the other hand, regarding the evaluation of (1) extendibility (on paper), a standard score (3 points) or more of the typical paint was obtained, but the average score of the diluent 3 having the highest content of the ethanol and the polyvinyl alcohol was the lowest. In this respect, from the viewpoint of combining the volatility and a film formability, it is considered that there is an advantage that when the diluent 3 is used, the diluent 3 stretches and spreads to the extent of the typical paint, and a clean and uniform film can be quickly formed.

From the above results, it has been confirmed that all of the diluent 1 to the diluent 3 have the good characteristics equivalent to or better than those of the typical paint by relatively evaluating that the film of the paint does not extend too much when smeared on the drawing paper and stretches and spreads as much as the typical paint, that the film of the finished paint is neatly formed without being blurred as compared with the typical paint, and that the film of the finished paint is uniform and has less irregularities.

Then, among the three diluents, the diluent 3 having these characteristics in a well-balanced manner was determined to be the most preferable, and in the following [Test Example 2], the diluent 3 was used as the diluent.

### Test Example 2

Evaluation of the Cosmetic as the Paint Material:

For the cosmetics A to P shown in Tables 1 to 9 above, various properties of the paint material were compared and evaluated using the diluent 3 selected in Test Example 1 as the diluent.

First, similar to Test Example 1, 3 g of the cosmetic and 3 g of the diluent were kneaded with the spatula for five minutes to form the mixture, and the cosmetic was formed into the paint. In addition, 0.2 g of the cosmetic formed into the paint and 0.2 g of the water (the tap water) were dissolved on the pallet to prepare the evaluation sample.

As a result of the above operation, the cosmetic C, the cosmetic E, and the cosmetic F were separated from the diluent, and the diluent was not compatible with the cosmetic, such that the paint could not be formed (consequently, it could not be dissolved in the water and applied to the drawing paper). In addition, the cosmetic G was also found to be slightly separated from the diluent, and when the cosmetic G was dissolved in the water and smeared on the drawing paper, using the cosmetic G as the paint was difficult. Accordingly, it was determined that the cosmetic C, the cosmetic E, the cosmetic F, and the cosmetic G were not suitable as the paint material, and the subsequent evaluations were not performed.

With respect to whether the cosmetic can be formed into a paint or not, when four or more oily components are contained in the top five places based on the all component information of the cosmetic shown in Tables 2 to 9, it is considered that the cosmetic contains so many oily components that the entire surface area of the powder such as the extender powders is covered with the oily component, and forming the paint is considered to be difficult. In addition, all of the cosmetics that were difficult to be formed into the paint had the creamy dosage form, and were soft enough to be recognized as in a slurry form when touched with finger.

On the other hand, when the contained components are arranged in the descending order of the content, there is no problem in forming the paint or the like for the cosmetic A, the cosmetic B, the cosmetic D, and the cosmetic H to the cosmetic P, which are the cosmetic having three or less oily components in the top five places and one or more extender powders in the top three places. From the above, 14 evaluation items (see the above for the contents of the evaluation items) of the (1) to (14) below were evaluated on the evaluation samples of the cosmetic A, the cosmetic B, the cosmetic D, and the cosmetics H to P according to the criteria shown in FIG. 1 (when the typical paint got 3 points). The results are shown in Table 12 (for the cosmetic A, the results in the case of using the diluent 1 and the diluent 2 were also described as a reference).

(Evaluation Items)

(1) Extendibility (on paper)

(2) Spread well (on paper)

(3) Quick-drying property

(4) Color development

(5) Transparency

(6) Over-coating (on paper)

(7) Peeling properties after drying

(8) Smoothness of film (formed on paper)

(9) Secondary adhesion

(10) Fixability of film (formed on paper)

(11) Smell

(12) Toning

(13) Solubility in water

(14) Solubility in water after solidification

(Results)

TABLE 12

| | Evaluation items (point: average score) | | | | | | | | | | | | | | |
|---|---|---|---|---|---|---|---|---|---|---|---|---|---|---|---|
| | (1) | | (2) | | (3) | (4) | | (5) | | (6) | | (7) | | (8) | |
| Cosmetic A* | 3.42 | ◯ | 3.08 | ◯ | 4.58 ⊙ | 2.08 | Δ | 5 | ⊙ | 2.83 | ◯ | 3 | ◯ | 3.33 | ◯ |
| Cosmetic A** | 3.25 | ◯ | 3.17 | ◯ | 4.75 ⊙ | 2.08 | Δ | 5 | ⊙ | 2.75 | ◯ | 3 | ◯ | 3.33 | ◯ |
| Cosmetic A | 3 | ◯ | 3.5 | ⊙ | 4.92 ⊙ | 2.08 | Δ | 5 | ⊙ | 2.92 | ◯ | 3.08 | ◯ | 3.5 | ⊙ |
| Cosmetic B | 2.92 | ◯ | 3.25 | ◯ | 4.92 ⊙ | 2.67 | ◯ | 4.62 | ⊙ | 3.42 | ◯ | 3.17 | ◯ | 3.25 | ◯ |
| Cosmetic D | 2.67 | ◯ | 3.17 | ◯ | 4.83 ⊙ | 2.83 | ◯ | 5 | ⊙ | 3.42 | ◯ | 3.08 | ◯ | 3.17 | ◯ |
| Cosmetic H | 3.25 | ◯ | 3.58 | ⊙ | 4.75 ⊙ | 2.5 | ◯ | 4.92 | ⊙ | 3.42 | ◯ | 3.17 | ◯ | 3.5 | ⊙ |
| Cosmetic I | 3.25 | ◯ | 3.33 | ◯ | 4.83 ⊙ | 1.67 | Δ | 5 | ⊙ | 3 | ◯ | 3 | ◯ | 3.5 | ⊙ |
| Cosmetic J | 3.08 | ◯ | 3.33 | ◯ | 4.83 ⊙ | 2.42 | Δ | 4.75 | ⊙ | 3.33 | ◯ | 3 | ◯ | 3.5 | ⊙ |
| Cosmetic K | 3.08 | ◯ | 3.33 | ◯ | 4.5 ⊙ | 2.33 | Δ | 5 | ⊙ | 3.25 | ◯ | 3 | ◯ | 3.5 | ⊙ |
| Cosmetic L | 3.42 | ◯ | 3.42 | ◯ | 4.58 ⊙ | 3 | ◯ | 4.25 | ⊙ | 3.42 | ◯ | 3 | ◯ | 3.42 | ◯ |
| Cosmetic M | 3.25 | ◯ | 3.5 | ⊙ | 4.75 ⊙ | 2.83 | ◯ | 4.83 | ⊙ | 3.5 | ⊙ | 2.92 | ◯ | 3.25 | ◯ |
| Cosmetic N | 2 | Δ | 2.75 | ◯ | 4.17 ⊙ | 3 | ◯ | 4.75 | ⊙ | 2.42 | Δ | 3.25 | ◯ | 1.92 | Δ |
| Cosmetic O | 3.75 | ⊙ | 3.42 | ◯ | 4.83 ⊙ | 2.92 | ◯ | 4.58 | ⊙ | 3.25 | ◯ | 3 | ◯ | 3.08 | ◯ |
| Cosmetic P | 3.75 | ⊙ | 3.58 | ⊙ | 4.92 ⊙ | 2.92 | ◯ | 4.17 | ⊙ | 3.42 | ◯ | 3 | ◯ | 3.33 | ◯ |

| | Evaluation items (point: average score) | | | | | | | | | | | | |
|---|---|---|---|---|---|---|---|---|---|---|---|---|---|
| | (8) | | (9) | | (10) | | (11) | | (12) | | (13) | | (14) |
| Cosmetic A* | 3.33 | ◯ | 2.75 | ◯ | 3.08 | ◯ | 3.5 | ⊙ | 2.58 | ◯ | 3 | ◯ | 3 ◯ |
| Cosmetic A** | 3.33 | ◯ | 2.67 | ◯ | 3.25 | ◯ | 3.58 | ⊙ | 2.58 | ◯ | 2.92 | ◯ | 3 ◯ |
| Cosmetic A | 3.5 | ⊙ | 2.67 | ◯ | 3.08 | ◯ | 3.58 | ⊙ | 2.58 | ◯ | 3.08 | ◯ | 3 ◯ |
| Cosmetic B | 3.25 | ◯ | 2.42 | Δ | 3.08 | ◯ | 3.5 | ⊙ | 3.17 | ◯ | 3.08 | ◯ | 3 ◯ |
| Cosmetic D | 3.17 | ◯ | 2.33 | Δ | 3.17 | ◯ | 3.5 | ⊙ | 3.08 | ◯ | 2.5 | ◯ | 3 ◯ |
| Cosmetic H | 3.5 | ⊙ | 2.25 | Δ | 3.25 | ◯ | 3.5 | ⊙ | 3.17 | ◯ | 3.08 | ◯ | 3 ◯ |
| Cosmetic I | 3.5 | ⊙ | 2.83 | ◯ | 3.25 | ◯ | 3.58 | ⊙ | 3.08 | ◯ | 3.33 | ◯ | 3 ◯ |
| Cosmetic J | 3.5 | ⊙ | 3.42 | ◯ | 3.33 | ◯ | 3.5 | ⊙ | 3.17 | ◯ | 3 | ◯ | 3 ◯ |
| Cosmetic K | 3.5 | ⊙ | 3 | ◯ | 3.08 | ◯ | 3.5 | ⊙ | 3.08 | ◯ | 3.08 | ◯ | 3 ◯ |
| Cosmetic L | 3.42 | ◯ | 3.25 | ◯ | 3.42 | ◯ | 3.5 | ⊙ | 3.25 | ◯ | 3 | ◯ | 3 ◯ |
| Cosmetic M | 3.25 | ◯ | 2.58 | ◯ | 3.33 | ◯ | 3.42 | ◯ | 3 | ◯ | 2.75 | ◯ | 3 ◯ |
| Cosmetic N | 1.92 | Δ | 1 | X | 1.83 | Δ | 3.42 | ◯ | 3.08 | ◯ | 2.83 | ◯ | 3 ◯ |
| Cosmetic O | 3.08 | ◯ | 2.25 | Δ | 3.25 | ◯ | 3.42 | ◯ | 3.08 | ◯ | 3.17 | ◯ | 3 ◯ |
| Cosmetic P | 3.33 | ◯ | 2.67 | ◯ | 3.25 | ◯ | 3.5 | ⊙ | 3 | ◯ | 3.17 | ◯ | 3 ◯ |

As shown in Table 12, some of the evaluated cosmetics were evaluated as “A,” for (4) color development and (9) secondary adhesion (for the cosmetic N, (1) extendibility (on paper), (6) over-coating (on paper), (8) smoothness of film (formed on paper), (10) fixability of film (formed on paper) is “A,” and (9) “secondary adhesion” is “x” will be described later), but the evaluation results were mostly good as a whole, and accordingly, it can be determined that the present cosmetic paint has the properties not much different from those of the typical watercolor paint.

Additionally, the items (1) to (14) are not essential characteristics for the paint and are merely confirmed to be relatively excellent and poor with respect to the typical paint, and the fact that the evaluation results of these items including the items (4) color development and (9) secondary adhesion are poor with respect to the typical paint does not mean that the cosmetic paint cannot be applied as the paint.

Here, for the (4) color development, some of the cosmetics (the cosmetic paints) that are evaluated as the paint this time are slightly inferior to the commercially available typical watercolor paint (the typical paint), because the content of the pigment blended in the powdery cosmetic itself produced for being smeared on the human skin may be lower than that in the typical paint.

However, regarding the (3) quick-drying property and the (5) transparency, the film of the drawing traces finished by the cosmetic paint has the transparency unique to the cosmetic paint, and comments of the panelists indicate that the film has the touch feeling unique to the cosmetic paint, which is not found in the typical paint and creates a preferable impression. From the above, although the color development is slightly inferior, it is considered that the cosmetic paint can be regarded as the paint, and a unique feeling of depiction can be presented by the touch with the transparency instead of the color development.

In addition, (9) secondary adhesion was slightly inferior to that of the typical paint in some cases. The reason is considered to be the fact that the powders such as the extender powders blended in the cosmetic is relatively large as compared with the typical paint having a powder with the particle diameter of substantially the pigment grade.

This is expected to be a state in which large powders contained in the cosmetic paint cannot enter the irregularities on the paper surface and adhere to the drawing paper surface as the film component, unlike the typical paint that enters the irregularities on the drawing paper surface in a soaked manner. Accordingly, it is considered that the film is easily entangled by a physical frictional force, and the secondary adhesion is lowered.

In addition, as described above, it has been confirmed that the (3) quick-drying property and the (5) transparency are evaluated to be better than the typical paint, and there are an advantage that a delicate design can be expressed by a light touch due to the transparency and another advantage that the drawing is easy due to the quick-drying property just like acrylic paint such that the drawing can be continued without waiting for the paint to dry.

Further, the evaluation of the (11) smell was also mostly better than that of the typical paint. The reason for this is that, although there are products with no smell and products with smell, the diluent itself does not have the smell unique to the watercolor paint, and further, the cosmetic paint formed by the cosmetic with smell is given a smell derived from the cosmetic and has the good smell unique to the cosmetic that does not give any discomfort.

Additionally, the cosmetic N containing the pearl powder as the largest content among all the components had a relatively low score in each item of (1) Extendibility (on paper), (6) over-coating (on paper), (8) smoothness of film (formed on paper), and (10) fixability of film (formed on paper), and a low score with the average score of 1 for (9) secondary adhesion.

This result is considered to be caused by the powder being caught on the brush tip because the pearl powder with larger particle diameter is contained in the largest amount among the components constituting the cosmetic. When the brush tip touches the paper, the large powder attached to the outer surface of the brush tip adheres to the paper, and the large powder is clogged between the brush tip at an initial stage of stretching and spreading, and the small powder is also clogged at the brush tip to be caught.

Thereafter, since only the component of the liquid form stretches and spreads to be sucked into the drawing paper, and the fluidity of the liquid component of the brush tip also decreases, it is considered that the extendibility of the paint is felt to decrease. From the above, it is considered that there is a bias in a localization of the powder in the drawing traces that extendibility on the drawing paper, (6) over-coating (on paper) becomes difficult, and the unevenness of the film of the drawing traces after drying also occurs. As a result, it is considered that (8) smoothness of film (formed on paper) is also affected, and (9) secondary adhesion is lowered and (10) fixability of film (formed on paper) is also lowered due to the lumps of the powder in which the drawing traces after drying are localized.

Additionally, the typical paint is usually used by putting the paint on the pallet, then drying and solidifying the paint. In order to verify whether the same use method for the typical paint is possible for the cosmetic paint as described above, (7) peeling properties after drying and (14) solubility in water after solidification were evaluated. As a result, as shown in Table 12, the paint was comparable to the typical paint, the peeling properties from the pallet and a poor solubility in the water were not confirmed, and it is confirmed that the paint could be used at the same level.

### INDUSTRIAL APPLICABILITY

The present invention can be advantageously used as a means for applying the cosmetic to be discarded as the paint material or the paint, and has a high industrial applicability.

1. A paint material comprising a cosmetic comprising:
extender powders and a coloring material, wherein
the paint material includes a cosmetic having three or less oily components in top five places when contained components are arranged in a descending order of a content.

2. The paint material comprising the cosmetic according to claim 1, wherein one or more of the extender powders in the top three places are contained when contained components are arranged in a descending order of a content.

3. The paint material comprising the cosmetic according to claim 2, wherein the extender powders included in the top three places is at least one selected from a group consisting of a mica, a talc, a synthetic phlogopite, a silica, and a barium sulfate.

4. The paint material comprising the cosmetic according to claim 1, wherein a pearl powder is not contained in the top three places when the contained components are arranged in the descending order of the content.

5. The paint material comprising the cosmetic according to claim 1, wherein the cosmetic is at least one selected from a group consisting of an eye shadow, a blusher, a body powder, a face powder, a bleaching powder, a skincare powder, a loose powder, a presto powder, and a foundation.

6. A diluent for a paint material that is for diluting the paint material comprising a cosmetic, the diluent comprising:
a water of 49.5 to 89.0 mass % with respect to the entire diluent;
a lower alcohol of 10.0 to 35.0 mass % with respect to the entire diluent; and
a polyvinyl alcohol of 0.5 to 15.0 mass % with respect to the entire diluent.

7. The diluent for the paint material according to claim 6, wherein the lower alcohol is an ethanol.

8. A paint comprising a mixture obtained by mixing:
the paint material comprising the cosmetic according to claim 1; and
the diluent for the paint material comprising:
a water of 49.5 to 89.0 mass % with respect to the entire diluent;
a lower alcohol of 10.0 to 35.0 mass % with respect to the entire diluent; and
a polyvinyl alcohol of 0.5 to 15.0 mass % with respect to the entire diluent.

9. (canceled)
